(19) United States
(12) Patent Application Publication
(10) Pub. No.: US 2010/0144633 A1
(43) Pub. Date: Jun. 10, 2010

(54) CHLOROPLAST EXPRESSION OF MEMBRANE PROTEINS

(76) Inventor: Henry Daniell, Winter Park, FL (US)

Correspondence Address: Timothy H. Van Dyke, 390 No. Orange Avenue, Suite 2500, Orlando, FL, 32801, US

(21) Appl. No.: 12/611,920

(22) Filed: Nov. 3, 2009

Related U.S. Application Data
(60) Provisional application No. 61/110,957, filed on Nov. 3, 2008.

(51) Int. Cl. A61K 38/16; C12N 15/82; A01H 5/00; C07H 21/04; C12P 21/06

(52) U.S. Cl. 514/12; 435/320.1; 800/298; 800/320.1; 800/320.2; 800/320; 800/320.3; 800/312; 800/317.2; 800/306; 800/317.3; 800/317.4; 800/305; 800/309; 800/314; 536/23.6; 435/468; 435/69.1

(57) ABSTRACT

Disclosed herein are chloroplast transformation vectors constructed to enable expression and hyperaccumulation of membrane proteins in chloroplasts. Another embodiment relates to plants transformed with such vectors. Another embodiment relates to seeds and other plant tissues transformed with such vectors. Another embodiment relates to a method of increasing expression of membrane proteins in chloroplasts including transforming a plant cell with vectors described herein.

### CROSS-REFERENCE TO RELATED APPLICATIONS

This application claims priority to U.S. Provisional application No. 61/110,957 filed Nov. 3, 2008. The teachings of this application are incorporated herein.

### GOVERNMENT INTEREST

The presently disclosed subject matter was made with U.S. Government support under USDA grant no. 3611-21000-017-00D, NIH 5R01 grant no. GM 63879-06 and NIH R01 grant no. GM61893. Thus, the U.S. Government has certain rights in the presently disclosed subject matter

### INTRODUCTION

Chloroplasts are highly complex organelles that perform a vast array of essential metabolic processes in plants and algae, including photosynthesis, amino acid and lipid metabolism, and secondary product synthesis. The biogenesis and differentiation of chloroplasts is dependent upon expression of genes encoded in the chloroplast and the nuclear genomes. The majority of nucleus-encoded chloroplast proteins are synthesized in the cytoplasm and imported into the organelle via the TOC-TIC translocation systems of the chloroplast envelope (Inaba and Schnell, 2008). In many cases, these proteins are further targeted to subcompartments of the organelle (e.g. the thylakoid membrane and lumen or inner envelope membrane) by additional targeting systems that function downstream of the import apparatus (Schunemann, 2007).

Much progress has been made in understanding the molecular mechanism of TOC-TIC function and in subsequent targeting of proteins to the thylakoid membrane. The TOC-TIC system consists of multi-subunit complexes within the outer and inner envelope membranes (Inaba and Schnell, 2008). These complexes physically associate to mediate preprotein recognition by binding preprotein transit peptides in the cytoplasm and provide direct transport of polypeptides from the cytoplasm to the chloroplast stroma via linked protein conducting channels. Nucleus- and chloroplast-encoded proteins are targeted from the stroma to thylakoids by at least four protein trafficking systems (Schunemann, 2007). These systems all correspond to those found in gram-negative bacteria and presumably were conserved from the original endosymbiont during chloroplast evolution.

In contrast to the protein import and thylakoid targeting systems, our knowledge of the pathways and molecular mechanisms of protein targeting and integration at the inner envelope membrane (IM) are very limited. The IM contains a complex array of proteins, including enzymes involved in lipid synthesis, the production of secondary products for plant defense and cellular signaling, and transporters that mediate the exchange of metabolites and the import of nucleus encoded proteins into the organelle (Block et al., 2007). As such, knowledge of the biogenesis of the IM is central to understanding the metabolic and communication networks that link chloroplasts with other cellular activities.

The bulk of IM proteins are nucleus-encoded in vascular plants with only one or two possible exceptions (Ferro et al., 2003; Ferro et al., 2002; Froehlich et al., 2003). At least two pathways for IM targeting have been proposed for nucleus encoded proteins that initially engage the TOC-TIC system for import from the cytoplasm. The first or so-called ‘stop-transfer’ pathway is directly coupled to the process of protein import at the envelope (Brink et al., 1995; Knight and Gray, 1995; Li et al., 1992). The stop-transfer models predict that proteins are directly integrated into the IM during translocation through the TIC translocon. In this scenario, the protein-conducting channel of the TIC complex would sense the presence of transmembrane helices and allow lateral diffusion of the helices into the lipid bilayer. The second pathway is proposed to function independent of the protein import process. In this so-called ‘post-import’ pathway, IM proteins are fully imported into the chloroplast stroma via the TOC-TIC system and inserted into the IM from the stroma via an unidentified translocon (Lubeck et al., 1997; Li and Schnell, 2006; Tripp et al., 2007; Chiu and Min Li, 2008). The most detailed evidence in support of the post-import pathway has come from studies using pre-atTic40, a single-pass transmembrane protein that functions as a cochaperone in the import apparatus (Chou et al., 2006). Pre-atTic40 normally is nucleus-encoded and is imported into the organelle after synthesis in the cytoplasm. Previous studies have shown that preatTic40 targeting to the IM involves a soluble intermediate that inserts into the IM from the chloroplast stroma after import from the cytoplasm (Li and Schnell, 2006; Tripp et al., 2007). Targeting to the IM involves two-step proteolytic processing that removes the transit peptide in the stroma and an additional N-terminal sequence at the IM.

### BRIEF DESCRIPTION OF THE DRAWINGS

FIG. 1. Chloroplast transformation vector, transformation and transgene integration. (A, B) Schematic representation of the chloroplast flanking sequence used for homologous recombination, probe DNA sequence (0.81 kb) obtained by digesting with BamHI and BglII and expected product of untransformed and transgenic lines with pLDutrpre-atTic40-His (digested with SmaI). (C) First round of selection and primary transplastomic shoots (D) Second round of selection. (E) Regenerated shoots on rooting medium for the third round of selection; all rounds of selection contained spectinomycin (500 mg/l); (F, G) PCR analysis of the transgenic plants using 3P/3M and 5P/2M respectively, for evaluation of site specific integration of the transgene cassette into the chloroplast genome. (WT, untransformed wild type; T1 to T4 transplastomic lines, P, positive control; M, 1 kb plus DNA ladder). (H) Southern blot hybridized with the flanking sequence probe showing homoplasmy (T1 to T4, T0 transplastomic lines; WT, untransformed plant). (I) Southern blot analysis of T1 transplastomic lines. (J) Phenotypes of a transgenic (TC59) and untransformed (WT) plant grown in green house. (K, L) Untransformed (WT) and transgenic (TC59) seeds germinated on MSO medium containing spectinomycin (500 mg/l) showing maternal inheritance.

FIG. 2. Phenotype and fertility of untransformed and transplastomic lines. (A) Transplastomic plants showing normal biomass and flowering. (B) Stamens, (C) Stigmas and (D-F) Longitudinal sections of the flower (WT, wild type; TC42 & TC59, transplastomic lines). (G-I) Transverse sections of the ovary showing normal development (WT, wild type; TC42 & TC59, transplastomic lines). (J, K) Wild type (WT) and transplastomic plant (TC59) showing normal fertilization and pod development.

FIG. 3. Expression and localization of pre-atTic40-His in transgenic tobacco. Leaf total lysates (10 μg protein) from wild type (WT) and transgenic tobacco lines TC42 and TC59 were resolved by SDS-PAGE and transferred to nitrocellulose membrane. (A) Imido black stained nitrocellulose filter of protein samples. The arrow indicates the position of atTic40-His. (B) Immunoblot of the filter in panel A with anti-His6 antibodies. Ly, total leaf lysate; M, alkaline extracted membrane fraction; S,alkaline extracted soluble fraction. The positions of molecular size standards are shown at the left of the figures.

FIG. 4. Pre-atTic40 is localized to chloroplast membranes. Chloroplasts were isolated from leaves of wild type (WT) and transgenic tobacco line TC59. Purified chloroplasts were extracted with alkaline carbonate to separate soluble and membrane components. Protein samples (10 μg) were resolved by SDS-PAGE and transferred to nitrocellulose membrane. Upper panel, imido black stained nitrocellulose filter of fractions from wild type (WT) and transgenic tobacco (TC59) leaves. Ly, total leaf lysate; PI, membrane pellet fraction; Sn, soluble fraction; T, total chloroplast fraction; M, alkaline extracted chloroplast membrane fraction; S, alkaline extracted chloroplast soluble fraction. Lane 1 contains a phosphorimage of in vitro translated (IVT) pre-atTic40-His, int-atTic40-His, and atTic40-His. Lower panel, immunoblot of the samples in panel A with anti-His₆ serum to detect the expressed atTic40-His. The positions of molecular size standards are shown at the right of the figure.

FIG. 5. Localization and functional assembly of atTic40-His at the chloroplast inner envelope in transgenic tobacco. (A) Immunofluorescence microscopic analyses of WT and TC59 chloroplasts. Isolated chloroplasts were imaged with anti-Hiss antiserum and goat anti-rabbit fluorescein IgG to detect atTic40-His. Chlorophyll autofluorescence is shown as an indicator of the position of the thylakoid membranes. Scale bar=5.0 μm. (B) atTic40-His fractionates with chloroplast inner membrane vesicles. Chloroplast membranes were separated by sucrose gradient centrifugation and fractions corresponding to inner membranes (IM) and thylakoid (Thyl) membranes (2.5 μg protein) were resolved by SDS-PAGE and immunoblotted with antisera to the proteins indicated at the left of the figure. (C) atTic40-His attains the correct topology in the inner membrane. Isolated inside-out IM vesicles were treated with thermolysin (T-lysin) in the presence or absence of Triton X-100 for the times indicated. The presence of atTic40-His, Tic110 and IEP37 was detected by immunoblotting with the indicated specific antibodies. (D) atTic40-His associates with other Tic components. Detergent soluble chloroplast extracts from WT, TC42 and TC59 tobacco were immunoprecipitated with anti-Tic110 serum. Immunoblot of total chloroplast extract (St), unbound fraction (Ft) and immunoprecipitates (El) with anti-Tic110, anti-Hsp93, anti-His₆ sera.

FIG. 6. Proliferation of the inner membrane in at Tic40-His transgenic tobacco. (A) Transmission electron micrographs of wild type and pre-atTic40-His transgenic lines TC42 and TC59. Samples of intact leaf tissue were fixed, stained and observed by transmission electron microscopy. The center four panels show chloroplasts from regions of leaf tissue from wild type (WT) and transgenic lines TC59 and TC42 (scale bar=1.0 μm). The uppermost and lowermost panels show higher magnifications of the boxed regions (scale bar=0.1 μm). The positions of the outer envelope membrane (OM), the inner envelope membrane (IM), the thylakoids (Th), the stroma (St), plastoglobule (PG), the cytoplasm (Cy), and mitochondrion (Mt) are labeled. (B) Immunoblots of serially diluted protein extracts from wild type and TC59 transgenic chloroplasts. Protein extracts of the amounts indicated at the top of the figure were resolved by SDSPAGE, transferred to nitrocellulose and immunoblotted with antisera to the proteins indicated at the left of the figure.

### DETAILED DESCRIPTION

The present invention stems from the inventors' research in elucidating the hypothesis that a subset of proteins are targeted to the IM independent of the protein import apparatus. The inventors realize that work supporting this hypothesis demonstrates that these proteins would successfully integrate into the IM if their genes were introduced into the chloroplast genome and the proteins were expressed in the stroma. The possibility of expressing IM proteins from the plastid genome provides a definitive experimental approach to test the post-import model. Furthermore, a plastid expression system for IM proteins opens up the possible use of the IM as a target for the expression of recombinant membrane proteins of agronomic and biotechnological interest.

The IM is a common feature of plastid types in all plant tissues, and the maternal inheritance of plastids in all crop species eliminates the concerns associated with nuclear transgenes. The transgenic chloroplast system has been used to express several soluble proteins, including vaccine antigens and human blood proteins (Verma & Daniell, 2007; Kamarajugadda & Daniell, 2006). Similarly, transgenic chloroplasts have been used to express several proteins to confer herbicide, insect, disease resistance or drought or salt tolerance (Daniell et al., 2005).

In one embodiment, the invention pertains to the successful demonstration of expressing IM proteins in chloroplasts by engineering the gene encoding pre-atTic40 into the plastid genome of tobacco. It is demonstrated herein that plastid-encoded pre-atTic40 is properly processed, targeted and successfully assembled into a functional complex at the IM. atTic40 accumulates to 15% of total chloroplast protein, resulting in a massive proliferation of the IM. Despite these dramatic changes in chloroplast ultrastructure, transgenic lines exhibited growth and fertility similar to control plants. This embodiment provides definitive evidence in support of the post-import pathway and establishes the potential of chloroplast genetic engineering to stably express membrane proteins within the organelle.

A membrane protein is a protein molecule that is attached to, or associated with the membrane of a cell or an organelle. More than half of all proteins interact with membranes. Biological membranes consist of a phospholipid bilayer and a variety of proteins that accomplish vital biological functions. Structural proteins are attached to microfilaments in the cytoskeleton which ensures stability of the cell. Cell recognition proteins allow cells to identify each other and interact. Such proteins are involved in immune response, for example. Membrane enzymes produce a variety of substances essential for cell function. Membrane receptor proteins serve as connection between the cell's internal and external environments. Finally, transport proteins play an important role in the maintenance of concentrations of ions. These transport proteins come in two forms: carrier proteins and channel proteins. Carrier proteins are involved in using the energy released from ATP being broken down to facilitate active transport and ion exchange. These processes ensure that useful substances are able to enter the cell and that toxic substances are pumped out of the cell.

Embodiments of the present invention pertain to the novel expression of membrane proteins in plastids. Disclosed herein are chloroplast transformation vectors constructed to enable expression and hyperaccumulation of membrane proteins in chloroplasts. Another embodiment relates to plants transformed with such vectors. Another embodiment relates to seeds and other plant tissues transformed with such vectors. Another embodiment relates to a method of increasing expression of membrane proteins in chloroplasts including transforming a plant cell with vectors described herein.

A plant remnant may include one or more molecules (such as, but not limited to, proteins and fragments thereof, minerals, nucleotides and fragments thereof, plant structural components, etc.) derived from the plant in which the protein of interest was expressed. Accordingly, a composition pertaining to whole plant material (e.g., whole or portions of plant leafs, stems, fruit, etc.) or crude plant extract would certainly contain a high concentration of plant remnants, as well as a composition comprising purified protein of interest that has one or more detectable plant remnants. In a specific embodiment, the plant remnant is rubisco.

According to a further embodiment, the invention pertains to a stable plastid transformation and expression vector which comprises an expression cassette comprising, as operably linked components in the 5′ to the 3′ direction of translation, a promoter operative in said plastid, a selectable marker sequence, a heterologous polynucleotide sequence coding for membrane protein, transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome.

Methods, vectors, and compositions for transforming plants and plant cells are taught for example in WO 01/72959; WO 03/057834; and WO 04/005467. WO 01/64023 discusses use of marker free gene constructs.

Proteins expressed in accord with certain embodiments taught herein may be used in vivo by administration to a subject, human or animal in a variety of ways. The pharmaceutical compositions may be administered orally or parenterally, i.e., subcutaneously, intramuscularly or intravenously, though oral administration is preferred.

Oral compositions produced by embodiments of the present invention can be administrated by the consumption of the foodstuff that has been manufactured with the transgenic plant producing the plastid derived therapeutic protein. The edible part of the plant, or portion thereof, is used as a dietary component. The therapeutic compositions can be formulated in a classical manner using solid or liquid vehicles, diluents and additives appropriate to the desired mode of administration. Orally, the composition can be administered in the form of tablets, capsules, granules, powders and the like with at least one vehicle, e.g., starch, calcium carbonate, sucrose, lactose, gelatin, etc. The preparation may also be emulsified. The active immunogenic ingredient is often mixed with excipients which are pharmaceutically acceptable and compatible with the active ingredient. Suitable excipients are, e.g., water, saline, dextrose, glycerol, ethanol or the like and combination thereof. In addition, if desired, the compositions may contain minor amounts of auxiliary substances such as wetting or emulsifying agents, pH buffering agents, or adjuvants. In a preferred embodiment the edible plant, juice, grain, leaves, tubers, stems, seeds, roots or other plant parts of the pharmaceutical producing transgenic plant is ingested by a human or an animal thus providing a very inexpensive means of treatment of or immunization against disease.

In a specific embodiment, plant material (e.g. lettuce material) comprising chloroplasts capable of expressing a membrane protein is homogenized and encapsulated. In one specific embodiment, an extract of the plant material is encapsulated. In an alternative embodiment, the plant material is powderized before encapsulation.

In alternative embodiments, the compositions may be provided with the juice of the transgenic plants for the convenience of administration. For said purpose, the plants to be transformed are preferably selected from the edible plants consisting of tomato, carrot and apple, among others, which are consumed usually in the form of juice.

According to another embodiment, the subject invention pertains to a transformed chloroplast genome that has been transformed with a vector comprising a heterologous gene that expresses a membrane protein.

Of particular present interest is a transformed chloroplast genome that has been transformed with a vector comprising a heterologous gene that expresses a CTB-Pins polypeptide. In a related embodiment, the subject invention pertains to a plant comprising at least one cell transformed to express a peptide as disclosed herein.

Reference to specific polypeptide sequences herein (such as but not limited to, known or yet to be characterized membrane proteins) relate to the full length amino acid sequences as well as at least 12, 15, 25, 50, 75, 100, 125, 150, 175, 200, 225, 250 or 265 contiguous amino acids selected from such amino acid sequences, or variants thereof.

Preferably, naturally or non-naturally occurring polypeptide variants have amino acid sequences which are at least about 55, 60, 65, or 70, preferably about 75, 80, 85, 90, 96, 96, or 98% identical to the full-length amino acid sequence or a fragment thereof. Percent identity between a putative polypeptide variant and a full length amino acid sequence is determined using the Blast2 alignment program (Blosum62, Expect 10, standard genetic codes).

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of polypeptide can be found using computer programs well known in the art, such as DNASTAR software. Whether an amino acid change results in a biologically active polypeptide can readily be determined by assaying for native activity.

Reference to genetic sequences herein refers to single- or double-stranded nucleic acid sequences and comprises a coding sequence or the complement of a coding sequence for polypeptide of interest. Degenerate nucleic acid sequences encoding polypeptides, as well as homologous nucleotide sequences which are at least about 50, 55, 60, 65, 60, preferably about 75, 90, 96, or 98% identical to the cDNA may be used in accordance with the teachings herein polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of nucleic acid sequences which encode biologically active polypeptides also are useful polynucleotides.

Variants and homologs of the nucleic acid sequences described above also are useful nucleic acid sequences. Typically, homologous polynucleotide sequences can be identified by hybridization of candidate polynucleotides to known polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each homologous sequences can be identified which contain at most about 25-30% basepair mismatches. More preferably, homologous nucleic acid strands contain 15-25% basepair mismatches, even more preferably 5-15% basepair mismatches.

Species homologs of polynucleotides referred to herein also can be identified by making suitable probes or primers and screening cDNA expression libraries. It is well known that the Tm of a double-stranded DNA decreases by 1-1.5° C. with every 1% decrease in homology (Bonner et al., J. Mol. Biol. 81, 123 (1973). Nucleotide sequences which hybridize to polynucleotides of interest, or their complements following stringent hybridization and/or wash conditions also are also useful polynucleotides. Stringent wash conditions are well known and understood in the art and are disclosed, for example, in Sambrook et al., MOLECULAR CLONING: A LABORATORY MANUAL, 2^{nd} ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a polynucleotide of interest or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Relevant articles on genetic sequences is provided: proinsulin (Brousseau et al., Gene, 1982 March; 17(3):279-89; Narrang et al, Can J Biochem Cell Biol. 1984 April; 62(4):209-16; and Georges et al, Gene 27 (2), 201-211 (1984); and CTB (Shi et al, Sheng Wu Hua Hsueh Tsa Chih 9 (No. 4), 395-399 (1993).

According to another embodiment, the invention pertains to a method of producing a membrane protein containing composition, the method including obtaining a stably transformed plant which includes a plastid stably transformed with an expression vector which has an expression cassette having, as operably linked components in the 5′ to the 3′ direction of translation, a promoter operative in a plastid, a selectable marker sequence, a heterologous polynucleotide sequence coding for polypeptide pertaining to a membrane protein, transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome; and homogenizing material of said stably transformed plant to produce homogenized material.

Embodiments of the invention demonstrate for the first time that membrane proteins can be successfully transcribed from the chloroplast genome, translated, processed and targeted to the inner envelope membrane. The utilization of pre-atTic40-His was used in conjunction with the study described in the examples below. However, the results described herein demonstrate the ability to utilize chloroplast transformation for the purpose of expressing the broader class of membrane proteins. The following is a non-exhaustive list of references that describe a non-limiting list of membrane proteins that may utilized in accordance with the teachings herein. U.S. Patent Publications 20090263403; 20090246227; 20090233853; 20090220536; 20090175912; 20060078572; 20050208123 and 20030044901. The teachings of these and any other references cited in this application are incorporated herein in their entirety to the extent not inconsistent with the teachings herein.

### Example 1

### Chloroplast Vector Design

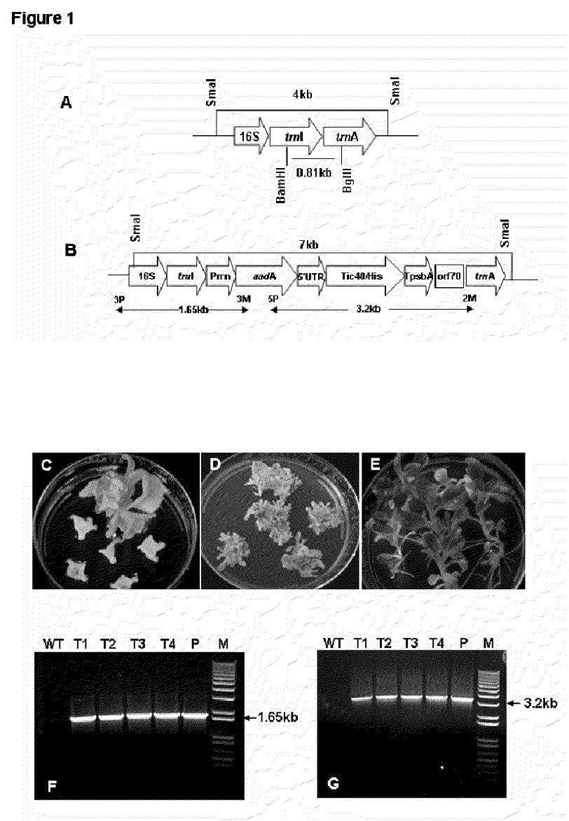

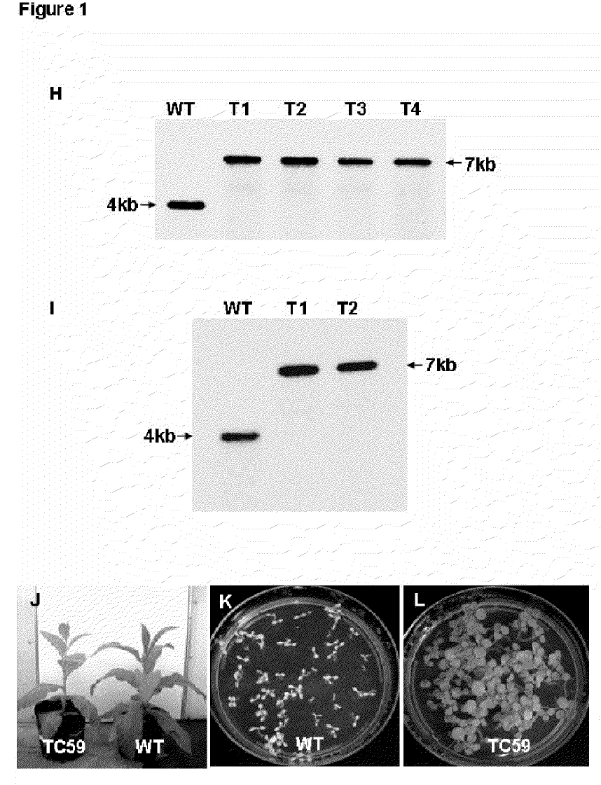

The pre-atTic40-His construct used in this study includes a C-terminal 6-histidine tag and a 76 amino acid N-terminal transit peptide that is normally required for import from the cytoplasm. The transit peptide is removed during import and targeting by a twostep process involving the stromal processing peptidase (SPP) and an unknown peptidase at the IM that is related to bacterial type I signal peptidases (Li and Schnell, 2006; Tripp et al., 2007). The pLD-utr-pre-atTic40-His vector used in this study for chloroplast transformation (FIG. 1B) is based on the universal chloroplast vector concept that has been used successfully in our laboratory (Verma and Daniell, 2007; Verma et al., 2008) for the expression of many transgenes into the tobacco chloroplast genome. The genes of interest are integrated into the spacer region between the trnI and trnA genes through homologous recombination of the flanking sequences between the transformation vector and the native chloroplast genome. This site of integration has several unique advantages (Daniell et al., 2004, Verma et al., 2008). The constitutive promoter 16S Rrna ribosomal promoter regulates the expression of aadA (aminoglycoside 3 adenyltransferase) gene. The psbA promoter and 5 UTR containing several ribosomal binding sites were engineered to enhance transcription and translation of the pre-at-Tic40-His gene. The transcript was stabilized by incorporating the psbA3 untranslated region.

### Example 2

### Transgene Integration into the Chloroplast Genome

Transplastomic plants were obtained as described previously (Daniell 1997; Daniell et al., 2004; Verma et al., 2008). Several shoots emerged from leaves 3-6 weeks after bombardment with gold particles coated with pLD-utr-pre-at-Tic40-His plasmid in the first round of selection (FIG. 1C). The second round of selection advanced shoots towards homoplasmy (FIG. 1D) and the third round of selection in root induction medium (FIG. 1E) established independent transgenic lines. PCR analysis using two sets of primers, 3P/3M and 5P/2M confirmed the transgene integration and site-specific integration of transgenes into chloroplast. As illustrated in FIG. 1B, the 3P primer annealed to the native chloroplast genome upstream of the site of integration and 3M primer lands on the aadA gene producing a 1.65 kb PCR product, while the 5P and 2M lands on aadA gene and trnA coding sequences respectively, which produced a 3.2 kb PCR product. All the transplastomic lines produced respective PCR products while no amplification was observed in untransformed lines (FIGS. 1F and G).

### Example 3

### Homoplasmy

Southern blot analysis was performed to confirm site specific integration of the pLD-utr-pre-atTic40-His cassette into the chloroplast genome and to determine homoplasmy (plants containing only transformed chloroplast genomes). Total plant DNA was isolated from the rooted plants and digested with the enzyme SmaI, which should generate a 4 kb fragment in untransformed (FIG. 1A) or a 7 kb fragment in transplastomic lines (FIG. 1B), when hybridized with a 0.81 kb flanking sequence probe. The [32P]-labeled trnI-trnA probe hybridized with 4 kb and 7 kb fragments in DNA from untransformed and transplastomic lines, respectively, confirming the correct insertion site of the transgenes between the trnI and trnA spacer region (FIG. 1H). Furthermore, the absence of a 4 kb fragment in the transgenic lines confirmed that homoplasmy has been achieved (within the levels of detection), even in transplastomic lines in the T0 generation. Transplastomic lines were transferred to jiffy pellets and kept under high humidity initially for two weeks, and then moved to the green house. Southern blot analysis of T1 transplastomic lines confirmed that all the transgenic lines maintained homoplasmy (FIG. 1I).

### Example 4

### Phenotypic Evaluation and Fertility of Transgenic Lines

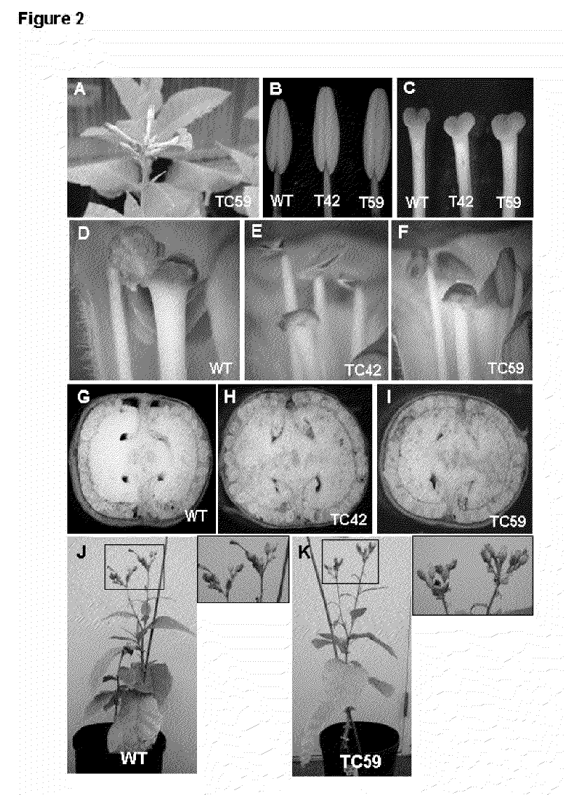

The phenotypes of transplastomic lines appeared to be normal (FIG. 1J). Transgenic seeds germinated and grew into green plants while wild type plants were bleached on spectinomycin containing medium (FIGS. 1K & L). The lack of transgene segregation suggests that transgenic lines are maternally inherited to their progeny. Plant 8 phenotype and fertility were investigated in untransformed and transgenic lines. Previous studies have shown that transgenic protein reduces plant fertility when it is toxic or adversely affects cellular or chloroplast metabolism. For example, male sterile plants with shorter stamens and no viable pollen were produced when B-keto thiolase was expressed in transgenic chloroplasts (Ruiz & Daniell, 2005). However, pre-atTic40-His transplastomic lines exhibited normal growth and fertility compared to untransformed plants (FIG. 2A-K). Both untransformed and transplastomic lines showed similar length of stamens, abundance of pollen grains and normal floral development, fertilization and pod development (FIG. 2A-K). Photosynthesis and plant pigments were not significantly different between untransformed and transplastomic lines (data not shown).

### Example 5

### Expression and Localization of Pre-atTic40-His in Transgenic Tobacco

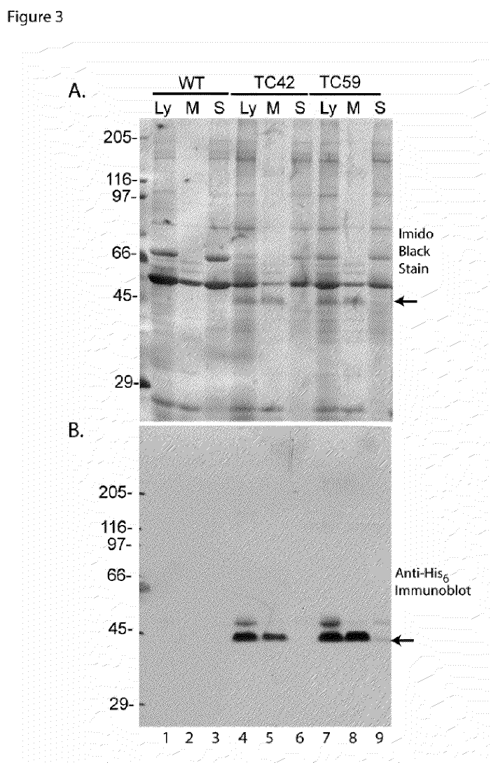

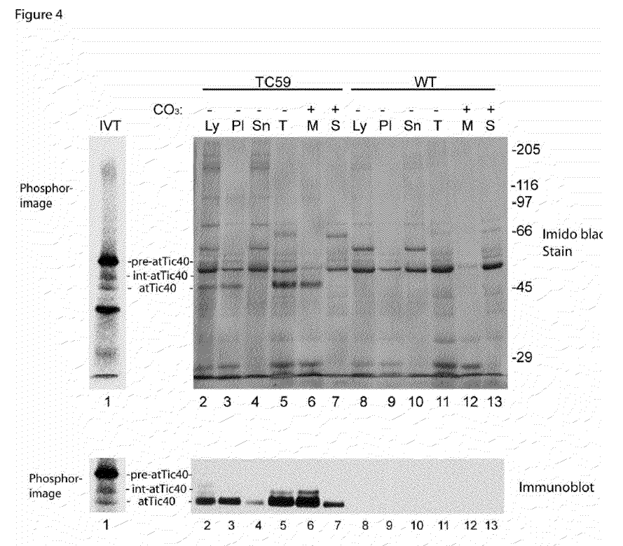

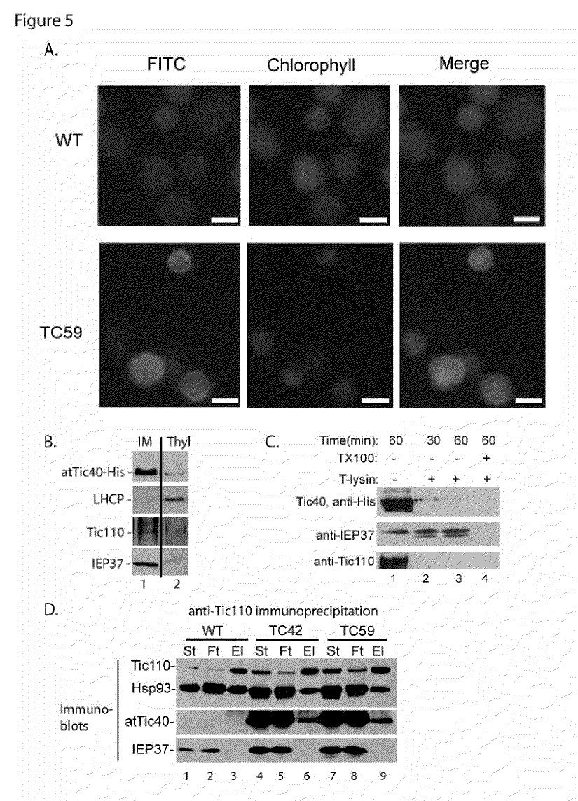

SDS-PAGE and immunoblot analysis of total leaf extracts confirmed expression of the pre-atTic40-His protein in two of the transgenic lines (TC42 and TC59, FIG. 3). A major 45 kDa polypeptide is present in TC42 and TC59 extracts (FIG. 3A, lanes 4 and 7, arrow). This polypeptide reacts with anti-His6 antibodies (FIG. 3B), confirming that it corresponds to atTic40-His. The atTic40-His polypeptide co-sediments with the membrane fraction of the leaf extracts, consistent with the membrane localization of atTic40 (FIG. 3, lanes 5 and 8). An immunoreactive band is not observed in extracts from untransformed plants (FIG. 3B, lanes 1-3). In order to confirm that pre-atTic40-His was expressed within chloroplasts, chloroplasts were isolated from transgenic line TC59 and untransformed tobacco plants. The 45 kDa imido black-stained band that reacted with the anti-His6 antibody was enriched in chloroplast fractions, confirming localization within this organelle (FIG. 4, compare lane 2 with 5). After extraction of the chloroplast samples with alkaline carbonate, the majority of atTic40-His remained with the membrane fraction, confirming membrane integration (FIG. 4, compare lanes 6 and 7). Pre-atTic40 is normally processed in two steps to generate mature atTic40 (Li and Schnell, 2006; Tripp et al., 2007). The first processing step is performed by the stromal processing peptidase during import from the cytoplasm to remove residues 1-42 of the Nterminal transit peptide (Li and Schnell, 2006). This generates a stromal intermediate (int-atTic40) that is further processed to mature atTic40 at the inner envelope membrane during the integration process (Li and Schnell, 2006). Comparison of chloroplastexpressed pre-atTic40-His in line TC59 to in vitro translated markers corresponding to pre-atTic40-His, int-atTic40-His and mature atTic40-His demonstrates that the vast majority of the transgenic protein had been processed to mature atTic40-His (FIG. 4, compare lanes 1 and 2). Although no pre-atTic40-His was detected, a minor species corresponding to int-atTic40-His was observed (FIG. 4, lower panel, lanes 5 and 6), suggesting that the transgenic protein was processed in two steps similar to pre-atTic40 imported from the cytoplasm. The proper targeting of atTic40-His is supported by the observations that it is membrane associated and processed to its mature form. To confirm these data, we examined the suborganellar localization of atTic40-His directly. AtTic40-His exhibited a peripheral localization pattern in immunofluorescence microscopy using the anti-His6 antibody in isolated chloroplasts (FIG. 5A). This pattern was distinct from the internal chlorophyll autofluorescence signal exhibited by thylakoid membranes and is consistent with localization to the envelope. To confirm that the immunofluorescence pattern corresponds to the IM and not thylakoids, we separated isolated chloroplast membranes by sucrose gradient centrifugation and probed the distribution of atTic40-His in fractions enriched in IM and thylakoid proteins. FIG. 5B shows that atTic40 is highly enriched in membrane fractions containing the IM markers, Tic110 and IEP37. This localization is distinct from LHCP, a thylakoid membrane marker. Taken together, these data confirm that pre-atTic40-His is synthesized, processed and properly targeted to the inner envelope membrane when expressed from the plastid genome. AtTic40 contains a single transmembrane helix at its N-terminal region, and the bulk of the protein extends into the stroma (Chou et al., 2003). In isolated inside-out inner membrane vesicles, the hydrophilic domain is exposed and therefore sensitive to protease digestion (Li and Schnell, 2006). To confirm that overexpressed atTic40-His attains the correct topology, isolated inner membrane vesicles were digested with thermolysin and the sensitivity of inner membrane proteins was examined by immunoblotting (FIG. 5C). As controls, we examined the sensitivity of two additional endogenous inner membrane proteins. Tic110 contains a short N-terminal membrane anchor with the bulk of the protein exposed to the stroma and is therefore susceptible to protease digestion in IM vesicles (Inaba et al., 2003). IEP37 is anchored at its C-terminus and extends into the intermembrane space between the outer and inner envelope (Motohashi et al., 2003). In isolated IM vesicles, IEP37 is largely insensitive to protease digestion because the bulk of the polypeptide is located within the vesicle lumen.

As expected, atTic40-His and the endogenous control, Tic110, are sensitive to protease treatment of inside-out vesicles (FIG. 5C, compare lanes 1, 2 and 3). In contrast, IEP37 is largely resistant to protease treatment (FIG. 5C, compare lanes 1, 2 and 3; Motohashi et al., 2003). In addition to full-length IEP37, a slightly smaller polypeptide is also present after digestion due to the partial cleavage of the short C-terminal tail of the protein. The resistance of IEP37 to proteolysis is not due to intrinsic stability because it was digested when the membrane barrier was disrupted by non-ionic detergent (FIG. 5C, compare lanes 3 and 4). Therefore, we conclude that atTic40-His attains the correct topology when expressed in the stroma and inserted into the inner envelope. As a final step to confirm that atTic40-His obtained its native structure; we examined its ability to interact with partner proteins at the inner envelope. atTic40 functions as a membrane-anchored co-chaperone within the TIC translocon of the envelope protein import apparatus. It aids in coordinating the transfer of preproteins from the translocon to the stromal chaperone machinery during the protein import process (Chou et al., 2006). Previous studies have demonstrated that Tic40 interacts with a TIC complex containing Tic110 and the stromal Hsp93 chaperone (Chou et al., 2006; Inaba et al., 2005). To test if atTic40-His could interact with this complex in transgenic tobacco, transgenic chloroplasts were treated with a covalent cross-linker to stabilize protein-protein interactions, dissolved with non-denaturing detergents, and subjected to immunoprecipitation with anti-Tic110 antibodies. FIG. 5D demonstrates that atTic40-His assembles into a TIC complex containing endogenous Hsp93 and Tic110 that is similar to those previously identified in pea and Arabidopsis (lanes 4 and 7) (Nielsen et al., 1997; Chou et al., 2003). These interactions are specific because another abundant protein, IEP37, was not detected in the immunoprecipitates. These results demonstrate 12 that a transgenic membrane protein can be expressed, targeted, integrated and properly folded into its native conformation when expressed from the chloroplast genome. Examination of the protein profiles of total leaf extracts or chloroplast fractions (FIGS. 3 and 4) indicated that atTic40-His was accumulating at levels that made it easily detectable by imido black staining. Quantification of the protein content in the TC59 samples indicated that atTic40-His accumulated to ˜15% of total chloroplast protein (FIG. 4 and data not shown), demonstrating the remarkable efficiency of the post-import pathway.

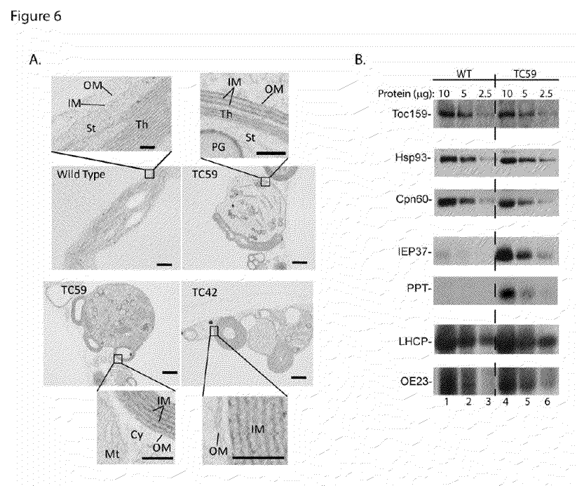

IM proteins normally constitute <1% of total chloroplast protein, raising the question of the effects of atTic40-His overexpression on the IM. To investigate this in more detail, we examined chloroplast ultrastructure in untransformed and transgenic lines by transmission electron microscopy (FIG. 6). Wild type chloroplasts exhibit typical envelope morphology containing tightly apposed outer and inner envelope membranes with roughly equivalent surface areas (FIG. 6, wild type). In contrast, chloroplasts from both transgenic lines exhibit a massive expansion of the inner envelope (FIGS. 6, TC59 and TC42). The membrane appears to invaginate and form stacks or whorls underneath the outer membrane.

The shape transgenic chloroplasts is altered due to such invaginations compared with the lens-shaped organelles observed in untransformed chloroplasts. Consistent with IM proliferation, the expression levels of other endogenous inner membrane proteins in line TC59 were significantly up regulated. IEP37 and the triose phosphate-phosphate translocator were upregulated ˜10 fold compared to untransformed plants as estimated by semi-quantitative immunoblotting (FIG. 6B). The levels of the outer envelope membrane marker, Toc159, and two stromal markers, hsp93 and cpn60 were not significantly different in transgenic and 13 control plants (FIG. 6B). Likewise, the thylakoid proteins, LHCP and OE23, appeared to be unchanged (FIG. 6B). Taken together, these data confirm that the IM specifically proliferates in response to overexpression of atTic40-His. The response includes the up regulation of other nucleus-encoded IM proteins, suggesting that IM proliferation involves a coordinated increase in the both lipid and protein components to provide increased membrane surface area to compensate for increased levels of atTic40.

### Discussion Related to Examples 1-5

The results of the foregoing examples provide definitive evidence for the existence of a post-import pathway for the targeting of nucleus-encoded proteins to the IM. Transfer of the gene encoding pre-atTic40-His to the plastid genome resulted in high level expression of the protein in the chloroplast stroma. Remarkably, pre-atTic40 was efficiently and properly inserted into the IM from its site of synthesis in the stroma, demonstrating that insertion of this class of proteins can occur independent of the protein import process. The molecular machinery mediating the post import pathway remains to be identified, but our results demonstrate the existence of a unique targeting pathway from the chloroplast stroma that is distinct from the conservative thylakoid protein targeting systems.

The chloroplast envelope is estimated to comprise ±1% of total chloroplast protein and <10% of chloroplast membrane surface area under normal conditions (Block et al., 2007). The fact that over-expressed atTic40-His alone can accumulate to ˜15% of total chloroplast protein and induce dramatic membrane proliferation provides key insights into the regulation of membrane biogenesis in this organelle. Chloroplasts are the major source of fatty acids in plant cells, and together with the ER, they generate the majority of cellular and chloroplast membrane lipids (Benning, 2008). The IM is the primary site of lipid synthesis and it plays a direct role in the generation of constituents for all three chloroplast membranes. Despite this interconnection, our data indicate that the biogenesis of the three chloroplast membrane systems is independently regulated. In the case of the inner envelope, membrane expansion is coupled to the levels of membrane protein synthesis.

One particularly interesting observation from the inventors' study was the apparent coordinate regulation of membrane expansion and membrane protein expression. The induction of membrane proliferation by overexpression of atTic40-His in the stroma resulted in the concomitant up regulation of the expression of other IM proteins, including IEP37, SAM-dependent methyltransferase, and the triose-phosphate phosphate translocator, an abundant polytopic metabolite transporter. These data suggest that the regulatory networks for lipid synthesis and protein expression that control IM biogenesis can respond to internal cues from the chloroplast. The up regulation of nuclear genes that code for other IM proteins and membrane proliferation should provide a novel system to understand signal transduction between chloroplast and nuclear genomes. Retrograde signaling factors studied so far include Mg-proto, redox signaling, inhibition of plastid gene expression or accumulation of ROS (Woodson and Chory, 2008) and no system is yet available to study the effect of nuclear genes in response to expression of a specific protein within chloroplasts.

The results presented herein also have significant implications for understanding membrane biogenesis and provide a first step in using chloroplast transformation as a means of expressing and accumulating high levels of native or foreign membrane proteins for structural studies or biomedical applications. Progress in understanding chloroplast biogenesis and gene expression have led to advances in the use of chloroplasts as highly efficient bioreactors for the expression of foreign proteins of biomedical and therapeutic interest. Foreign proteins have been shown to accumulate at levels up to 46% of the total leaf protein (DeCosa et al., 2001) and it is possible to produce up to 360 million doses of fully functional anthrax vaccine in one acre of tobacco (Koya et al., 2005). Vaccine antigens against bacterial (Koya et al., 2005; Daniell et al., 2001; Arlen et al., 2008; Tregoning et al., 2003), viral (Molina et al., 2004) and protozoan (Chebolu and Daniell, 2007) pathogens have been expressed in transgenic chloroplasts and results of successful challenges with toxins or pathogens have been demonstrated (Koya et al., 2005; Daniell et al., 2001; Arlen et al., 2008; Tregoning et al., 2003; Verma and Daniell, 2007; Kamarajugadda and Daniell, 2006). One goal of the inventors was to extend the success in expressing soluble foreign proteins in chloroplasts to include membrane proteins. Heterologous expression systems in prokaryotes for the expression of membrane proteins have been hampered by different synthesis, targeting, insertion and folding characteristics in their hosts. Adequate expression of membrane proteins continues to be a major challenge due the toxic effects which severely reduce cell growth product yields (Wagner et al., 2006; Wagner et al., 2007). Therefore, finding suitable expression systems for membrane proteins is highly desirable. The ability of transgenic chloroplasts to express membrane and soluble proteins makes them ideal targets for metabolic engineering and biotechnology applications. For example, localization of —tocopherol (vitamin E) synthesis in the IM (Arango and Heise, 1998) facilitates engineering of an essential dietary nutrient for human and animal health.

The high levels of atTic40-His expression with limited effects on organelle function or plant growth and development open the door for the use of transgenic chloroplasts to overexpress membrane proteins for various biomedical applications. Space for accommodation of over-expressed membrane proteins has been a major bottle neck and mechanisms that regulate membrane proliferation are poorly understood (Wagner et al., 2006). In contrast to the thylakoid membrane that is restricted to green tissues, the IM provides a target for constitutively expressed membrane proteins in all plant tissue types.

Maternal inheritance of genetically modified chloroplast genomes and the absence of any reproductive structures when foreign proteins are expressed in leaves, offer efficient transgene containment and facilitates their safe production in the field (Daniell, 2007). Therefore, transplastomic plants producing human therapeutic proteins have been tested in the field after obtaining USDA-APHIS approval (Arlen et al., 2007). These unique advantages make chloroplast an ideal bioreactor for expression of membrane proteins for biomedical applications

### Methods Related to Examples 1-5

Vector construction The pET-21d plasmid which contains pre-at-Tic40-His (Gen Bank accession #BT006595)(Li and Schnell, 2006) was digested with NcoI/SacI to subclone the pre-at-Tic40-His fragment. This pre-at-Tic40-His fragment was subcloned into NcoI/SacI digested site of pUC-utr-aphA6 plasmid by replacing aphA6 gene to generate pUC-utrpreat-Tic40-His. Further this plasmid was digested with EcoRI to remove the utr-preat-Tic40-His fragment and finally cloned into EcoRI digested and dephosphorylated chloroplast expression vector pLDCtV2. The final construct was designated as pLD-utrpreat-Tic40-His.

Regeneration of transplastomic plants Nicotiana tabacum var. Petit Havana was grown aseptically on hormone-free Murashige and Skoog (MS) agar medium containing 30 g/l sucrose. Sterile young leaves from plants at the 4-6-leaf stages were bombarded using gold particles coated with vector pLD-utrpreat-Tic40 and transplastomic plants were regenerated as described previously (Daniell, 1997; Daniell et al., 2004; Verma et al., 2008).

Confirmation of transgene integration by PCR and Southern blot Plant genomic DNA was isolated using Qiagen DNeasy plant mini kit from the spectinomycin resistant primary shoots. PCR analysis was performed to confirm transgenes integration in the inverted repeat regions of the chloroplast genome using two set of primers 3P/3M and 5P/2M, respectively (Daniell et al., 2001). The PCR reaction was performed as described previously (Daniell, et al., 2001; Verma et al., 2008). Leaf from the PCR positive shoots were again cut into small pieces and transferred on RMOP medium containing 500 mg/l spectinomycin for additional round of selection and subsequently moved to MSO (MS salts without vitamins and growth hormones) medium containing 500 mg/ml spectinomycin for another round of selection to generate homoplasmy. The Southern blot analysis was performed according to lab protocol (Kumar and Daniell, 2004). In brief, total plant genomic DNA (1-2 μg) isolated from third round of selection was digested with SmaI and separated on a 0.8% agarose gel and then transferred to a nylon membrane. The chloroplast flanking sequence probe was made by digesting pUC-18 Ct vector DNA with BamHI and Bg/II which generate a 0.81 kb probe (FIG. 1A). After labeling the probe with 32P [dCTP], the membrane was hybridized using Stratagene QUICK-HYB hybridization solution and protocol. Seeds collected from the Southern confirmed T0 seeds were germinated in vitro in spectinomycin containing medium. Finally they were transferred to pot and move to green house. Total DNA was isolated from the T1 plants and Southern blot was performed as described to see the transgenes inheritance using same flanking probe used above.

Phenotypic differences between wild type and transgenic lines To check the differences in phenotypes and fertility between wild type and transgenic lines, plants were raised in the green house. Floral parts were dissected from transgenic lines as well from wild plants. Leaf chlorophyll was quantified in 80% (v/v) acetone extracts by measuring the A663 and 645 nm and using the Arnon equations for total chlorophyll content (Arnon, 1949).

Chloroplast isolation, fractionation and immunoblotting Chloroplast isolation was performed as described previously (Smith et al., 2002). Chloroplasts were lysed by suspension in 50 mM HEPES-KOH, pH 7.5, 330 mM Sorbitol (HS buffer) to a concentration of 0.5-1 mg chlorophyll/ml and diluted with five volumes of 2 mM EDTA. The lysate was mixed vigorously and incubated on ice for 10 min. The samples were adjusted to 0.2 M NaCl and the membrane fraction was collected by centrifugation at 18,000 g for 30 minutes at 4° C. For alkaline extraction, the membrane pellet was re-suspended with a small volume of HS buffer and diluted with volumes of 0.2 M Na2CO3, pH 12. The samples were homogenized with a Teflon homogenizer (Kontes Glass Co. Vineland N.J.), incubated at room temperature for 10 min, and the membrane fraction was collected by centrifugation at 100,000 g for 15 min. The soluble fractions were removed and concentrated by precipitation in 20% trichloroacetic acid. Inner membrane and thylakoid membranes were separated from hypertonically lysed chloroplasts by linear sucrose gradients using the method of Keegstra and Yousif (1986).

Membrane vesicles (1 mg protein/ml) were treated with thermolysin at a ratio of 200 mg thermolysin/mg protein for 30 min on ice. Proteolysis was stopped with the addition of an excess of ice cold 10 mM EDTA. All samples were resolved by SDS-PAGE, transferred to nitrocellulose filters and immunoblotted with antisera using chemilumenescence detection.

Crosslinking of chloroplasts and co-immunoprecipitation experiments Isolated intact chloroplasts (200 μg chlorophyll) were resuspended in HS buffer to a concentration of 1 mg/ml chlorophyll and dithiobis(succinimidyl propionate) (DSP) was added to a final concentration of 0.5 mM. The crosslinking reaction was incubated on ice in dark for 15 min and quenched by adding glycine to 50 mM and continuing incubation for another 15 min. Crosslinked chloroplasts were re-isolated through percoll gradients, washed with HS buffer and dissolved with dissolving buffer (50 mM Hepes-KoH pH7.5, 150 mM NaCl, 1% triton X-100) with 1% PIC (protease inhibitor cocktail, Sigma) at 4° C. for 30 min with constant gentle shaking. After a 30-min centrifugation at 18,000 g, the supernatant was collected and incubated with 20 μg anti-Tic110 antibody for 2 hrs. Packed protein G agarose beads (20 μl, Santa Cruz Biotechnology, Inc.) were added and the incubation was continued overnight. The beads were recovered at 1000 g for 2 min and washed with 500 μl washing buffer (50 mM Hepes-KoH pH7.5, 150 mM NaCl, 0.5% triton 20×-100 and 0.1% PIC). Bound proteins were separated by SDS-PAGE and analyzed by immunoblotting.

Immunofluorescence and electron microscopy Immunofluorescence microscopy was performed as previously described (Schnell and Blobel, 1991) with anti-atTic110 primary and fluorescein-coupled goat anti-rabbit as secondary antibodies. Samples were viewed with a Nikon E-600 epifluorescence microscope (Nikon Instrument, Melville, N.Y.) equipped with an FITC-HYQ filter set (EX460-500, DM505, BA510-560). A Spot-RT camera system (Diagnostic Instrument, Sterling Heights, Mich.) was used for image capture.

For electron microscopy, tissue samples were fixed with 2.5% glutaraldehyde in 0.05 M sodium cacodylate, pH 7.4 under vacuum for 3 h and subsequently washed three times with 0.05 M sodium cacodylate, pH 7.4. Fixed samples were treated with 1% osmium tetroxide in 0.05 M sodium cacodylate, pH 7.4 for 2 h, and washed three times with 0.05 M sodium cacodylate, pH 7.4. The samples were dehydrated by the following treatments: incubation in 70% ethanol for 10 min, incubation in 100% ethanol for 10 min, and incubation twice in 100% propylene oxide for 15 min. EMbed812 embedding mixture (Electron Microscopic Sciences, Fort Washington, Pa.) was prepared according to the manufacturer's instructions. Dehydrated samples were infiltrated with ⅓ concentrated Mbed812 (in propylene oxide) for 3 h, ⅔ concentrated Embed812 overnight and 100% Embed812 for 1.5 h before being embedded in EMbed812 by incubation at 60 C for 24 h. 70 nm-sections of the samples were prepared and dried on 150-mesh copper grids and post-stained with uranyl acetate and lead citrate as previously described (Smith and Croft, 1991). The grids were dried and observed using a Philips-Tecnai 12 Transmission Electron Microscope.

### REFERENCES

Arango, Y., and Heise, K. P. (1998) Localization of alpha-tocopherol synthesis in chromoplast envelope membranes of Capsicum annuum L. fruits. J. Exp. Bot. 49: 1259-1262.
Arlen, P. A., Ding, Y., Singleton, M., Adamovicz, J. J., Daniell, H. (2008). Effective plague vaccination via oral delivery of plant cells expressing F1-V antigens in chloroplasts. Infect. Immun. 76: 3640-3650.
Arlen, P. A., Falconer, R., Cherukumilli, S., Cole, A., Cole, A. M., Oishi, K. K., and Daniell, H. (2007). Field production and functional evaluation of chloroplast derived interferon-2b. Plant Biotechnol. J. 5: 511-525.
Arnon, D. (1949). Copper enzymes in isolated chloroplasts. Polyphenoloxidase in Beta vulgaris. Plant Physiol. 24: 1-15.
Benning, C. (2008). A role for lipid trafficking in chloroplast biogenesis. Prog Lipid Res. 47: 381-389.
Block, M. A., Douce, R., Joyard, J., and Rolland, N. (2007). Chloroplast envelope membranes: a dynamic interface between plastids and the cytosol. Photosynth. Res. 92: 225-244.
Brink, S., Fischer, K., Klosgen, R.-B., and Flugge, U.-I. (1995). Sorting of nuclearencoded chloroplast membrane proteins to the envelope and the thylakoid membrane. J. Biol. Chem. 270, 20808-20815.
Chebolu, S., and Daniell, H. (2007). Stable expression of Gal/GaINAc lectin of Entamoeba histolytica in transgenic chloroplasts and immunogenicity in mice towards vaccine development for amoebiasis. Plant Biotechnol. J. 5: 230-239.
Chiu, C. C., and Min Li, H. (2008). Tic40 Is Important for Reinsertion of Proteins from the Chloroplast Stroma into the Inner Membrane. Plant J. [Epub ahead of print]
Chou, M. L., Chu, C. C., Chen, L. J., Akita, M., and Li, H. M. (2006). Stimulation of transit-peptide release and ATP hydrolysis by a cochaperone during protein import into chloroplasts. J. Cell Biol. 175: 893-900.
Chou, M. L., Fitzpatrick L. M., Tu, S. L., Budziszewski, G., Potter-Lewis, S., Akita, M., Levin, J. Z., Keegstra, K., and Li, H. M. (2003). Tic40, a membrane-anchored co-chaperonehomolog in the chloroplast protein translocon. Embo. J. 22: 2970-2980.22
Daniell, H. (1997). Transformation and foreign gene expression in plants mediated by microprojectile bombardment. Methods Mol. Biol. 62: 453-488.
Daniell, H. (2007). Transgene containment by maternal inheritance. Effective or elusive? Proc. Natl. Acad. Sci. USA 104: 6879-6880.
Daniell, H., Datta, R., Varma. S., Gray, S., and Lee, S. B. (1998). Containment of herbicide resistance through genetic engineering of the chloroplast genome. Nat. Biotechnol. 16: 345-348.
Daniell, H., Dhingra, A., and Ruiz, O. N. (2004). Chloroplast genetic engineering to improve agronomic traits. Methods Mol. Biol. 286: 111-37.
Daniell, H., Lee S. B., Panchal, T., and Wiebe, P. O. (2001). Expression of the native cholera toxin B subunit gene and assembly as functional oligomers in transgenic tobacco chloroplasts. J. Mol. Biol. 311: 1001-1009.
DeCosa, B., Moar, W., Lee, S. B., Miller, M., and Daniell, H. (2001). Overexpression of the Bt cry2Aa2 operon in chloroplasts leads to formation of insecticidal crystals. Nat. Biotechnol. 19: 71-74.
Dhingra, A., Portes, A. R., and Daniel, H. (2004). Enhanced translation of achloroplast expressed RbcS gene restores SSU levels and photosynthesis in nuclear antisense RbcS plants. Proc. Natl. Acad. Sci. USA. 101: 6315-6320.
Ferro, M., Salvi, D., Riviere-Rolland, H., Vermat, T., Seigneurin-Berny, D., Grunwald, D., Garin, J., Joyard, J., and Rolland, N. (2002). Integral membrane proteins of the chloroplast envelope: identification and subcellular localization of new transporters. Proc Natl Acad Sci USA 99, 11487-11492.
Ferro, M., Salvi, D., Brugiere, S., Miras, S., Kowalski, S., Louwagie, M., Garin, J., Joyard, J., and Rolland, N. (2003). Proteomics of the chloroplast envelope membranes from Arabidopsis thaliana. Mol Cell Proteomics 2, 325-345.
Froehlich, J. E., Wilkerson, C. G., Ray, W. K., McAndrew, R. S., Osteryoung, K. W., Gage, D. A., and Phinney, B. S. (2003). Proteomic study of the Arabidopsis thaliana chloroplastic envelope membrane utilizing alternatives to traditional two-dimensional electrophoresis. J Proteome Res 2, 413-425.
Inaba, T., Alvarez-Huerta, M., Li, M., Bauer, J., Ewers, C., Kessler, F., and Schnell, D. J. (2005). atTic110 Is Essential for the Assembly and Function of the Protein Import Machinery of Plastids. Plant Cell 17: 1482-96.
Inaba, T., Li, M., Alvarez-Huerta, M., Kessler, F., Schnell, D. J. (2003). atTic110 Functions as a Scaffold for Coordinating the Stromal Events of Protein Import into Chloroplasts. J. Biol. Chem. 278: 38617-3827.
Inaba, T., and Schnell, D. J. (2008). Protein trafficking to plastids: one theme, many variations. Biochem J 413, 15-28.
Kamarajugadda, S., and Daniell, H. (2006). Chloroplast-derived anthrax and other vaccine antigens: their immunogenic and immunoprotective properties. Expert Rev. Vaccines 5: 839-849.
Keegstra, K., and Yousif, A. E. (1986). Isolation and characterization of chloroplast envelope membranes. Methods Enzymol. 118, 316-325.
Knight, J. S., and Gray, J. C. (1995). The N-terminal hydrophobic region of the mature phosphate translocator is sufficient for targeting to the chloroplast inner envelope membrane. Plant Cell 7, 1421-1432.
Koya, V., Moayeri, M., Leppla, S. H., and Daniell, H. (2005). Plant-Based Vaccine: Mice Immunized with Chloroplast-Derived Anthrax Protective Antigen Survive Anthrax Lethal Toxin Challenge. Infect. Immun. 73: 8266-8274.
Kumar, S., and Daniell, H. (2004). Engineering the chloroplast genome for hyperexpression of human therapeutic proteins and vaccine antigens. Methods Mol. Biol. 267: 365-383.
Li, H. M., S. Perry and K. Keegstra. (1992). Targeting of proteins into and across the chloroplastic envelope. Membrane Biogenesis and Protein Targeting.
Li, M., and Schnell, D. J. (2006). Reconstitution of protein targeting to the inner envelope membrane of chloroplasts. J Cell Biol 175, 249-259.
Lubeck, J., Heins, L., and Soll, J. (1997). A nuclear-encoded chloroplastic inner envelope membrane protein uses a soluble sorting intermediate upon import into the organelle. J. Cell Biol. 137, 1279-1286.
Molina, A., Hervas-Stubbs, S., Daniell, H., Mingo-Castel, A. M. and Veramendi, J. (2004). High-yield expression of a viral peptide animal vaccine in transgenic tobacco chloroplasts. Plant Biotechnol. J. 2: 141-153.
Motohashi, R., Ito, T., Kobayashi, M., Taji, T., Nagata, N., Asami, T., Yoshida, S., Yamaguchi-Shinizaki, K., and Shinozaki, K. (2003). Functional analysis of the 37 kDa inner envelope membrane polypeptide in chloroplast biogenesis using a Ds tagged Arabidopsis pale-green mutant. Plant J. 34: 719-31.
Nielsen, E., Akita, M., Davila-Aponte, J., and Keegstra, K. (1997). Stable association of chloroplastic precursors with protein translocation complexes that contain proteins from both envelope membranes and a stromal Hsp100 molecular chaperone. EMBO J. 16, 935-946.
Schnell, D. J., G. Blobel, and Pain., D. (1991). Signal peptide analogs derived from two chloroplast precursors interact with the signal recognition system of the chloroplast envelope. J. Biol. Chem. 266, 3335-3342.
Schunemann, D. (2007). Mechanisms of protein import into thylakoids of chloroplasts. Biol Chem 388, 907-915.
Smith, M. D., Hiltbrunner, A., Kessler, F., and Schnell, D. J. (2002). The targeting of the atToc159 preprotein receptor to the chloroplast outer membrane is mediated by its GTPase domain and is regulated by GTP. J Cell Biol 159, 833-843. Tregoning, J. S., Nixon, P., Kuroda, H., Svab, Z., Clare, S., Bowe, F., Fairweather, N., Ytterberg, J., van Wijk, K. J., Dougan, G., and Maliga, P. (2003). Expression of tetanus toxin Fragment C in tobacco chloroplasts. Nucleic Acids Res. 31: 1174-1179.
Tripp, J., Inoue, K., Keegstra, K., and Froehlich, J. E. (2007). A novel serine/prolinerich domain in combination with a transmembrane domain is required for the insertion of AtTic40 into the inner envelope membrane of chloroplasts. Plant J. 52:824-838.
Verma, D., and Daniell, H. (2007). Chloroplast vector systems for Biotechnology applications. Plant Physiol. 145:1129-1143
Verma, D., Samson, N. P., Koya, V., and Daniell, H. (2008). A protocol for expression of foreign genes in chloroplasts. Nat. Protocols 3: 739-758.24
Wagner, S., Baars, L., Ytterberg, A. J., Klussmeie, A., Wagner, C. S., Nord, O., Nygren, P. Å., van Wijk, K. J., and de Gier, J. W. (2007). Molecular & Cellular Proteomics 6.9: 1527-1550.
Wagner, S., Bader, M. L., Drew, D., and de Gier, J. W. (2006). Rationalizing membrane protein overexpression. Trends. Biotechnol. 24: 364-371.
Woodson, J. D., and Chory, J. (2008). Coordination of gene expression between organellar and nuclear genomes. Nature Reviews Genetics 9: 383-395.25

For background on chloroplast transformation, plants and vectors, Applicant cites to the following US patent publication references. 2008024196, 20080189803, 20070124838, 20070124838. In addition, Applicant cites to U.S. application Ser. No. 11/915,666; filed Nov. 27, 2007. The teachings of all references cited herein are incorporated herein in their entirety to the extent not inconsistent with the teachings herein.

What is claimed is:

1. A plant composition comprising an immunologically effective amount of a membrane protein and a plant remnant, wherein the membrane protein is heterologous respective to source of plant remnant.

2. A stable plastid transformation and expression vector which comprises an expression cassette comprising, as operably linked components in the 5′ to the 3′ direction of transcription, a promoter operative in said plastid, a selectable marker sequence, a heterologous polynucleotide sequence coding for a membrane protein, transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome.

3. A vector of claim 2, wherein the plastid is selected from the group consisting of chloroplasts, chromoplasts, amyloplasts, proplastide, leucoplasts and etioplasts.

4. A vector of claim 2, wherein the selectable marker sequence is an antibiotic-free selectable marker.

5. A stably transformed plant which comprises a plastid stably transformed to express a heterologous polypeptide, or the progeny thereof, including seeds, wherein said heterologous polypeptide comprises at least 90 percent identity with a membrane protein.

6. A stably transformed plant of claim 5 which is maize, rice, grass, rye, barley, oat, wheat, soybean, peanut, grape, potato, sweet potato, pea, canola, tobacco, tomato, lettuce, carrot, melon, or cotton.

7. A stably transformed plant of claim 6 which is edible for mammals and/or humans.

8. A process for producing a membrane protein comprising: integrating a plastid transformation vector according to claim 2 into the plastid genome of a plant cell; growing a plant comprising said plant cell to thereby express said membrane protein; and obtaining expressed membrane protein, wherein obtaining comprises purifying said membrane protein, at least partially, respective to other proteins in said plant.

9. A plastid genome transformed to contain a polynucleotide having at least 90 percent identity to a membrane protein, said plastid genome configured so as to express said polynucleotide.

10. A method of inducing membrane proliferation in a plant cell comprising integrating a plastid transformation vector according to claim 2 into the plastid genome of a plant cell; and subjecting said plant cell under conditions to express said polynucleotide encoding said membrane protein.

11. The method of claim 10, wherein said membrane protein is atTic40-His.

12. A method of claim inducing expression of endogenous membrane proteins, said method comprising integrating a plastid transformation vector according to claim 2 into the plastid genome of a plant cell; and subjecting said plant cell under conditions to express said polynucleotide encoding said membrane protein.

13. The method of claim 12, wherein said membrane protein is atTic40-His.
